(19) United States
(12) Patent Application Publication
(10) Pub. No.: US 2019/0025276 A1
(43) Pub. Date: Jan. 24, 2019

(54) METHOD FOR PREDICTING THE BIOAVAILABILITY OF A RADIOELEMENT FOLLOWING CONTAMINATION, AND USES THEREOF

(71) Applicants: Commissariat à l'énergie atomique et aux énergies alternatives, Paris (FR); Orano Cycle, Courbevoie (FR)

(72) Inventors: Nina Griffiths, Gif Sur Yvette (FR); Anne Van Der Meeren, Evry Gregy Sur Yerres (FR)

(21) Appl. No.: 16/064,343

(22) PCT Filed: Dec. 22, 2016
(86) PCT No.: PCT/EP2016/082290

§ 371 Date: Jun. 20, 2018

(30) Foreign Application Priority Data
Dec. 23, 2015 (FR) 15 63214

(51) Int. Cl. G01N 33/487; G01N 33/94; G01N 33/50; G01N 33/84; G01T 1/161

(52) U.S. Cl. CPC G01N 33/48714; G01N 33/94; A61K 31/721; G01N 33/84; G01T 1/161; G01N 33/5008

(57) ABSTRACT

Method for predicting the bioavailability of a radioelement in an animal living organism following the contamination of the organism by the radioelement, the method comprising the steps of: (a) producing a gel imitating 5 the area of contamination in the animal living organism and in which the radioelement is uniformly distributed; (b) bringing the gel produced in the step (a) into contact with a solution imitating a biological fluid associated with the area of contamination in the animal living organism, then stirring the mixture; and (c) measuring the quantity of the radioelement in the solution at a given moment t, the measurement allowing the 10 prediction of the bioavailability of the radioelement in the animal living organism. The invention also relates to the use of such a method for identifying a molecule exhibiting chelating properties in relation to a given radioelement and/or for characterising the chelating properties of a molecule.

### TECHNICAL FIELD

The present invention is directed towards the general field of contamination, in particular human contamination, by radioelements.

More specifically, the present invention proposes a method for predicting the bioavailability of radioelements such as actinides further to contamination. This method uses a static compartment mimicking the contamination site which also corresponds to the retention site of the radioelement, and a dynamic compartment mimicking the biological fluid in contact with the contamination site and in which the radioelement may dissolve and be entrained for its removal.

The present invention also concerns the use of said method to identify a molecule capable of chelating radioelements and/or to characterize the chelating properties of said molecule.

### STATE OF THE PRIOR ART

Human contamination with radioelements such as actinides may occur during industrial activities related to the nuclear fuel cycle: mining of uranium, chemical purification and conversion of uranium, isotopic separation, production of nuclear fuel, reprocessing of spent nuclear fuel and recycling, remediation and dismantling of nuclear plants, or storage of nuclear waste or materials. It may also occur in research laboratories working in any nuclear-related field or involving the use of radioelements. This contamination can occur via inhalation, ingestion, injury and/or by contact.

Further to such a contamination, it must be possible to be able to assess the behaviour of the radioelements in the body, in order to evaluate the means available to limit the effects thereof on the body and the hazard level thereof. To do so, consideration must be given to their solubility: a soluble compound will be rapidly eliminated by the body whereas a compound that is not or only scarcely soluble will remain in the body for a longer time.

For this purpose, there exist different in vitro dissolution methods used for actinides and described by Ansoborlo et al, 1999 [1]. Schematically, these methods are based on the depositing of the compound to be tested on a membrane. The solubilised fraction is recovered via filtration of a buffer through the membrane after incubation of the membrane in the presence of this buffer. Another technique is based on incubation of particles in a buffer, followed by centrifugation to separate the particles from the dissolved fraction. Another system traps the particles between two membranes, and the solubilised fraction is collected in the outer compartment of the system. However, none of these techniques proves to be sufficiently reliable or reproducible.

One technique that can further be cited is based on the principle of diffusion of metallic species through a well-defined diffusion gel and accumulation thereof on an ion exchange membrane. This technique initially described by Davison and Zhang, 1994 [2] is called «Diffusive Gradient in Thin film» (DGT). It is notably employed to examine labile metals in natural waters, sediments and soils.

Additionally, dissolution tests of metallic particles chiefly in nanometric form are described in the literature, in media mimicking biological fluids. However, these tests are conducted under static conditions, therefore much removed from the conditions encountered in a living body and are essentially based on separation of particles/dissolved element.

In the pharmaceutical field, clearly differing from the field of contamination by radioelements, hydrogels such as agarose gel are already used to study drug diffusion. For example, Klose et al, 2009 [3] describe an in vitro method to evaluate the diffusion of a pharmaceutical drug. This method is based on a study of the diffusion of a pharmaceutical compound in a 0.1%-0.6% agarose gel. More particularly, once the agarose gel has solidified, a hole is pierced in the centre of the gel forming a reservoir in which a solution is placed comprising the compound to be characterized. Samples of the gel are taken at different distances from the reservoir and the amount of compound in the samples taken is evaluated. Hoang Thi et al, 2010 [4] reproduce this principle of the use of agarose gels to examine the diffusion of a pharmaceutical compound. Chaibva and Walker, 2007 [5] compare different methods to evaluate the in vitro release of a pharmaceutical drug, the gels employed being 20-30% agarose gels. Also known and used in the pharmaceutical field are compositions that can mimic a given biological compartment [6].

In the sphere of physiology, clearly differing from that of contamination by radioelements, in vivo measurements were made of absorptive capacity in the colon, chiefly movement of water and electrolytes (Na+, K+). Agarose has the advantage of allowing fluidic and ionic diffusion [7].

Finally, it is to be pointed out that the handling of radioelements and in particular of actinides such as plutonium (Pu) raises difficulties of technical nature. On account of the very strong specific activity of Pu isotopes (>10⁹ Bq/g), it is practically impossible, from a particle suspension, to conduct reproducible sampling in terms of activity. Inter-sample reproducibility is better for more soluble compounds. In addition, on account of the strong adsorption of Pu on any surface and chiefly on plastics, sampling must be conducted over a very short period of time in order to be reproducible.

As a result, tests using radioelements such as actinides must be performed very rapidly after preparation of the samples, which may generate uncertainties regarding reliability. In other words, on account of the specific activity of isotopes and their strong adsorption on surfaces it is very difficult to carry out reliable reproducible tests involving radioelements such as actinides.

There is therefore a true need for an in vitro method to predict or determine the bioavailability of radioelements in vivo further to human contamination, giving consideration to the specificities and constraints particular to radioelements, and not having the disadvantages of the methods known in the prior art.

### DESCRIPTION OF THE INVENTION

The method of the invention meets this need and additionally affords other advantages. The inventors have developed a method making it possible to predict the behaviour and hence the in vivo bioavailability of a radioelement further to contamination of a living being by this radioelement, said method being fast, relatively easy to implement, not requiring any special instrumentation except for measurement of the released radioelement. It is to be noted that the method is acellular i.e. it does not require the use of any cell or cell culture for implementation thereof.

The inventors propose a method involving two essential elements which are (1) a gel mimicking the contamination site (static compartment) containing the radioelement for which it is desired to determine bioavailability, and (2) a liquid in contact with the gel and into which the radioelement is able to be released, said liquid being subjected to agitation and mimicking the dynamic compartment in contact with the contaminated site.

As previously explained, the method of the invention allows evaluation of the capacity of a radioelement to leave the contamination site. If the examined radioelement incorporated in the gel does not pass into the dynamic compartment, then this means that it remains at the contamination site and is therefore insoluble and/or trapped therein and is therefore potentially hazardous.

By preparing a single gel solution, when implementing the invention, having a given composition containing the radioelement to be examined, and then fractionating the same, it is possible to guarantee very high reproducibility of the «quantity» notably in terms of mass and activity of the radioelement under consideration including if it is in particle form. In addition, this preparation can be used for soluble, moderately soluble or even insoluble compounds.

With the method of the invention, the composition of the static compartment and/or dynamic compartment can easily be changed thereby making it possible not only to test very numerous compositions mimicking different biological conditions but also to evaluate the influence of numerous parameters on the dissolution/bioavailability of a radioelement to be tested such as mass, composition of the retention compartments and pH. These properties, associated with the fact that the method of the invention offers the possibility of treating very numerous samples under the same conditions and at the same time (several hundred), mean that said method can be termed a high throughput technique.

Finally, the method of the invention uses volumes of gel (static compartment) or liquid in contact with this gel (dynamic compartment) that are relatively low, typically lower than 1 ml and 5 ml respectively, allowing a miniature predictive test to be obtained.

More particularly, the present invention concerns a method for predicting the bioavailability of a radioelement in a living animal body, further to contamination of this body by said radioelement, comprising the steps of:

a) preparing a gel in which said radioelement is uniformly distributed, said gel mimicking the contamination site in said living animal body;

b) placing the gel prepared at said step (a) in contact with a solution mimicking a biological fluid associated with the contamination site in said living animal body, then leaving the whole under agitation; and

c) measuring, at time t, the amount of said radioelement in said solution, said measurement allowing prediction of the bioavailability of said radioelement in said living animal body.

It is to be noted that the expressions «biological fluid» and «biological liquid» are used indifferently in the present description.

The term «bioavailability» in the present invention designates the proportion of radioelements having effective action at measurement time tin the living body compared with the total amount of radioelements to which the body is exposed at the time of contamination. In vitro, this bioavailability can be expressed as a proportion of radioelement released into the solution at time t from the gel prepared at step (a) compared with the total amount of radioelement initially placed in this gel when it was prepared. Therefore, the bioavailability of the radioelement at time t is calculated in accordance with following formula (I):

Bioavailability (at t)=(amount in solution at t)/(total initial amount)×100 (I)

By «living body» it is meant any living animal body and in particular a human being.

The present invention applies to any natural or artificial radioelement likely to contaminate a living body. It is specified that the terms «radioelement» and «radionuclide» are used indifferently in the present description. In one particular embodiment, said radioelement is an actinide. As more particular examples of actinides, for which it may be desired to predict bioavailability, mention can be made of uranium, plutonium, thorium, americium, curium, neptunium or any mixture thereof. In the present invention the examined radioelement can be in different chemical forms and in particular an oxide, nitrate, fluoride, or metallic, and/or in solid form whether or not soluble.

As previously indicated, the method of the invention uses a gel. The gels are generally formed from at least two constituents: a solution (hereafter called gel solution) which is a liquid «trapped» by a second compound which forms a three-dimensional net or three-dimensional network throughout the entire solution. This three-dimensional network is composed of compounds having colloidal properties able to form a matrix i.e. a solid continuous phase. Typically, a gel is a soft material, swollen with solution and capable of undergoing major deformation.

In the present invention, all the compounds with colloidal properties generally employed to prepare gel, particularly in biology, can be used in the invention. Advantageously, these compounds with colloidal properties are of organic nature: they are generally macromolecules which typically are molecules of relatively high molecular weight having a structure essentially formed of multiple repeat units derived, de facto or via design, from molecules of low molecular weight.

For example, as compounds having colloidal properties, it is possible to use macromolecules such as polysaccharides and in particular agarose, sucrose, sepharose, chitosan, xanthan, carrageenan, dextran, agar, alginate or one of the mixtures thereof. It is effectively possible to use mixtures of two different polysaccharides such as a mixture of carrageenan and agarose, and even more than two. Mention can also be made, as an example, of a polyacrylamide gel or derivative thereof formed by polymerization of an acrylamide or acrylamide derivative in the presence of a crosslinking agent.

Advantageously, when preparing the gel used in the method of the invention, the ratio of compound(s) having colloidal properties/gel solution (weight expressed in g/volume expressed in ml) is between 0.01 and 0.05, typically between 0.02 and 0.04. It is possible for example to prepare an agarose gel in which the agarose/gel solution ratio is 2.5% i.e. 0.25 g agarose/10 ml gel solution.

The gel used in the present invention has the property of mimicking the site contaminated by the radioelement. The notion «to mimic» means that the gel is a model representing the contaminated site or one of the elements (tissue or cells) contained in the contaminated site and in which the radioelement(s) may be retained. This model has physicochemical properties particularly in terms of pH, ion, protein and lipid concentrations comparable with or similar to those of the contaminated site element, and optionally has one or more constituent compound(s) characteristic of the contaminated site or said element, the gel differing however from the contaminated site through the absence of cells. The gel solution and/or additional compounds retained in the three-dimensional network of the gel provide the gel with its mimicking properties.

Therefore, the gel solution used in the method of the invention is typically an aqueous solution e.g. physiological saline solution composed of distilled water and sodium chloride (NaCl) diluted to a concentration of 8 to 9 g·L⁻¹, optionally completed with one or more salts and in particular one or more organic or inorganic salts. These salts and in particular these organic or inorganic salts are used to modify the ion concentration and pH of the solution. As examples of said organic or inorganic salts mention can be made of sodium chloride (NaCl), potassium chloride (KCl), sodium bicarbonate (NaHCO₃), sodium phosphate (Na₃PO₄), sodium hydrogen phosphate (Na₂HPO₄), sodium dihydrogen phosphate (NaH₂PO₄), potassium hydrogen phosphate (K₂HPO₄), potassium dihydrogen phosphate (KH₂PO₄), hydrochloric acid (HCl), magnesium chloride (MgCl₂), calcium chloride (CaCl₂), sodium sulfate (Na₂SO₄), sodium hydroxide (NaOH), potassium hydroxide (KOH), sodium acetate (CH₃COONa), sodium tartrate (Na₂C₄H₄O₆), sodium lactate (C(OH)(CH₃)COONa), sodium pyruvate (CH₃C(O)COONa), sodium citrate (Na₃C₆H₅O₂) and citric acid (C₆H₈O₇).

The mimicked contaminated site may be an element or compartment of a human body likely to be contaminated further to inhalation or ingestion of a radioelement or further to contact or injury in the presence thereof. Therefore, the contaminated site can be lung mucosa, buccal mucosa, gastric mucosa, cornea, dermis, liver (subsequent to the elimination process of a contaminating radioelement), a muscle, cartilage, bone, synovia (or synovial fluid), gastric juice, pulmonary mucus or surfactant and phagolysosome (or phagocytic vacuole) contained in phagocyte cells.

It is clear that to examine the bioavailability of a radioelement further to contamination e.g. via injury it is possible to implement the method of the invention using several gels each mimicking an element or compartment: muscle, cartilage, bone, synovia (or synovial liquid) all likely to be contaminated at the time of such injury, but also a phagolysosome further to recruitment of macrophages circulating towards the injured site.

As previously explained, the gel may have one or more constituent compounds characteristic of the mimicked contaminated site or one of the elements present at the mimicked contaminated site. This or these compounds mostly belong to the families of glycoproteins and glycosaminoglycans. This or these compounds are notably selected from among the group consisting of pepsin, lecithin, glycine, glucose, lysozyme, albumin, sodium taurocholate, maleic acid, transferrin, ferritin, fibrin, hyaluronic acid, glucosamine, fibronectin, laminin, mucin, keratins, collagens, chondroitin, osteopontin, hydroxyapatite and glutamic acid. Persons skilled in the art, in the light of their knowledge, are able to select the suitable compound(s) and the amount to be used in the gel as a function of the mimicked contaminated site. In particular, they may refer to the gels provided in the experimental section below and mimicking synovial fluid, cartilage, simple (ECM1) or more complex (ECM2) extracellular matrix. ECM2 includes collagen but also other compounds such as glucosamine (a building block of all monosaccharides) and hyaluronic acid, a glycosaminoglycan being the majority compound of this matrix. These two gels do not represent different tissues.

At step (a) of the method of the present invention, a gel is prepared in which the radioelement to be examined is uniformly distributed. By «uniformly distributed» it is meant that amount of radioelement in two identical, independently selected volumes of gel will be substantially the same. The notion of uniform distribution is opposed to the case in which the radioelement has accumulated at a given site of the gel such as a hollow forming a reservoir of radioelements.

The preparation step of the gel is dependent on the compounds employed, and is known in the corresponding field. It is generally performed by placing in solution the compounds having colloidal properties followed by formation of the gel. The gel may form spontaneously. For chemical compounds such as polysaccharides, it is generally necessary to heat and then cool the solution containing the compounds having colloidal properties. Heating ensures dispersion of the compounds in the solution and allows cleavage of some of the weak bonds existing between the different compounds which can then reorganize themselves to form a three-dimensional network. Gelling occurs on cooling of the solution. For example, regarding polyacrylamide gels, the formation of the gel requires the use of a crosslinking agent and acrylamide.

Advantageously the gel in which the radioelement is uniformly distributed can be prepared in a single step. For this purpose, it is desirable to add the radioelement directly when preparing the gel. In this case, the method will comprise a step to prepare a gel from at least one compound having colloidal properties, a solution such as previously defined (gel solution) and at least one radioelement.

As previously explained, the present invention applies to soluble radioelements as well as radioelements that are not or only scarcely soluble. Therefore, the placing in solution of a solid radioelement leads to a solution (complete dissolution of a soluble radioelement) or to a suspension (radioelement scarcely or not soluble).

In one particular embodiment, the radioelement in the form of a solution is taken up in a gel solution such as previously defined to which at least one compound having colloidal properties is added. This embodiment typically applies to soluble or moderately soluble radioelements. For example, a known activity (in Bq) of a radioelement is placed in a glass vial. After evaporation over a hot plate the radioelement is taken up in a solution that will be added to a gel solution. This preparation provides a solution of precise concentration which can be used as reference, the activity of the solution and the take-up volume being controlled.

As a variant, the radioelement is added in the form of a suspension to the gel solution containing at least one compound having colloidal properties. This variant notably applies to insoluble or scarcely soluble radioelements such as metallic oxides. Typically, a suspension of the radioelement is prepared in a glove box and the volume of suspension containing the desired activity is directly added to the gel solution already containing at least one compound having colloidal properties such as an agarose solution. In this variant, the volume of suspension added to the gel solution is 1% or lower of the volume of the gel solution.

When preparing the gel, it is evidently possible to add one or more characteristic constituent compounds such as previously defined. The latter can be added to the gel solution before, after or during the addition of the radioelement and/or to the gel solution before, after or during the addition of the compound having colloidal properties.

It is advantageous to impart a chosen morphology to the gel; this morphology often being imparted when preparing said gel. For this purpose, it is possible use special moulds corresponding to the shape that the user wishes to impart to the gel. It is also possible to model the gel, after preparation thereof, using adapted instruments such as blades. Typically, in the present invention, the composition comprising the gel solution, at least one compound having colloidal properties, the radioelement and optionally at least one characteristic constituent compound is poured, before any solidification, into a well of a multi-well plate conventionally used in biology or biotechnology. For example, a volume of composition of between 500 and 700 μl is placed in a well such as 3.9 cm² well of a 12-well plate.

Once the gel comprising the uniformly distributed radioelement is prepared in accordance with step (a) of the method of the present invention, it is possible to perform step (b) immediately after or, on the contrary, it can be delayed. Before implementation thereof, it is possible to store the gel prepared at step (a) in sealed wrapping to prevent drying and changes in the properties of the gel. Typically, it can be stored at 4° C. for a time not exceeding 36 h and in particular not exceeding 24 h.

At step (b) of the method of the present invention, the gel containing the radioelement and mimicking the contamination site is placed in contact with a solution mimicking a biological fluid associated with said contamination site in said living body.

By «biological fluid associated with the contamination site» it is meant a biological fluid with which the contamination site such as previously defined has in vivo exchanges such as fluid exchanges, nutrient exchanges and/or waste exchanges. This biological fluid may have in vivo continuous fluid contact with the contamination site i.e. it irrigates the contamination site. As a variant, this biological fluid is a fluid that circulates, passes or flows in vivo close to the contamination site. As examples of such biological fluids, mention can be made of blood, lymph, saliva, tears, aqueous humour of the anterior chamber (of the eye), interstitial fluid and sweat. In addition, for a given contamination site, several different biological fluids can be associated therewith. For example, for contamination via ingestion the biological fluid associated with the buccal mucosa, and via which the contaminant can be eliminated, can be either blood irrigating this mucosa or saliva.

Step (b) of the method of the invention uses a solution mimicking said biological fluid. Everything defined in the foregoing for the gel mimicking the contamination site applies mutatis mutandis to the solution mimicking the biological fluid associated with the contamination site. This solution therefore exhibits physicochemical properties in terms of pH, viscosity, ion, protein and lipid concentrations comparable with or similar to those of the mimicked biological fluid and may optionally comprise one or more characteristic constituent compounds of this fluid, the solution differing from the latter however through the absence of cells.

Advantageously, the solution of step (b) is an aqueous solution comprising one or more organic or organic salts selected from the group formed by sodium chloride (NaCl), potassium chloride (KCl), sodium bicarbonate (NaHCO₃), sodium phosphate (Na₃PO₄), sodium hydrogen phosphate (Na₂HPO₄), sodium dihydrogen phosphate (NaH₂PO₄), potassium hydrogen phosphate (K₂HPO₄), potassium dihydrogen phosphate (KH₂PO₄), hydrochloric acid (HCl), magnesium chloride (MgCl₂), calcium chloride (CaCl₂), sodium sulfate (Na₂SO₄), sodium hydroxide (NaOH), potassium hydroxide (KOH), sodium acetate (CH₃COONa), sodium tartrate (Na₂C₄H₄O₆), sodium lactate (C(OH)(CH₃)COONa), sodium pyruvate (CH₃C(O)COONa), sodium citrate (Na₃C₆H₅O₂) and citric acid (C₆H₈O₇).

As examples of constituent compounds able to be more particularly added to the solution of step (b), mention can be made of pepsin, lecithin, glycine, glucose, lysozyme, albumin, sodium taurocholate, maleic acid, transferrin, ferritin and fibrin.

Different solutions are known to those skilled in the art that can be used to mimic a biological fluid of a living body. For example, Marques et al, 2011 [6] describe the composition of several of these solutions that can be used in the present invention. As particular examples, a culture medium (RPMI 1640 or MEM type) with 10% addition of foetal calf serum, and the Gambles solution can be used to mimic blood and lung fluid respectively.

It is to be noted that, depending on the gel and associated solution, the composition of the gel solution and that of the solution used at step (b) can be the same or different. For example, the gel solution and the solution used at step (b) comprise one or more same or different salts.

The contacting of the solution and the gel at step (b) of the method can be carried out using any conventional technique allowing a gel to be placed in solution or to apply a solution to a gel. For example, when the gel is prepared in a well of a multi-well plate, the solution mimicking the biological fluid is distributed over the gel using a pipette so that the gel is covered with the solution. For example, a volume of between 2 and 3 ml of solution is added to a well e.g. a 3.9 cm² well of a 12-well plate containing the gel.

In addition, at step (b) and to mimic conditions existing in vivo between the contamination site and the biological fluid associated therewith, and in particular the dynamic mode of this contacting, the gel mimicking the contamination site and the solution mimicking this biological fluid are placed under agitation. Therefore, the term «whole» previously used for step (b) corresponds to the gel placed in contact with the solution. Advantageously, this agitation takes place at relatively low speed. By «relatively low speed» in the present invention it is meant a rotating speed of 250 rpm or less, in particular of 2 rpm or higher and in particular of between 5 and 150 rpm. More particularly, the multi-well plate in which at least one well contains a gel covered with solution such as previously defined is arranged on an agitator having a rotating speed of 5 rpm and placed in an incubator.

Similarly, the contacting and agitation at step (b) are conducted under conditions similar to those encountered in a living body, notably in terms of temperature and surrounding atmosphere. Advantageously, step (b) is performed in incubators conventionally used for cell culture i.e. an incubator with agitation having a temperature of approximately 37° C. (i.e. 37° C.±5° C. and in particular 37° C.±3° C.) the composition of the controlled atmosphere being humid air comprising 5% CO₂. It is also possible to perform step (b) under sterile conditions allowing study of the behaviour of the radioelement distributed within the gel in rich solutions i.e. lending themselves to bacterial or fungal contaminations over long periods of time.

Depending on the radioelement being studied in the method of the invention, it is within the reach of skilled persons without displaying an inventiveness to determine the technique(s) that can be used to measure, at time t, the amount of radioelement contained in the solution in contact with the gel, this corresponding to step (c) of the method of the present invention. The choice made will notably be function of the type of radiation emitted by the radioelement. As examples to illustrate the techniques that can be used for such measurement, mention can be made of spectrometry techniques in particular using gas detectors, semiconductor detectors such as germanium, cadmium telluride and silicon, inorganic scintillation detectors using sodium iodide, caesium iodide or bismuth germanate as scintillator, organic liquid scintillation detectors or organic solid scintillation detectors.

Also, the possible treatment conditions of this solution before this measurement step are dependent not only on the examined radioelement but also on the selected measurement technique. Skilled persons, without any inventiveness, are able to choose those that are best adapted to the radioelement and to the measurement technique. This also applies to possible treatment conditions of the gel, in particular to determine the total amount of radioelement to be used as set forth in the experimental section below.

It is to be noted that the measured time t is evaluated in relation to t₀ defined as the time at which the solution and gel are placed in contact at step (b).

The present invention also concerns the use of a method such as previously defined to identify a molecule having chelating properties towards a given radioelement and/or to characterize the chelating properties of a molecule.

The notion of «chelating properties» in the present invention means the capability of a natural or synthetic molecule to bind and form a stable complex with a radioelement such as an actinide. The formation of said complex allows trapping of the radioelement and prevention of undesired interactions by blocking normal reactivity of the radioelement. Said molecules are called «chelating agents» but also «decorporation agents» owing to their ability to cause decorporation of the radioelement i.e. excretion of the radioelement outside the living body via natural routes (urinary and faecal routes).

The method of the present invention offers several advantages and specificities particularly adapted for identification and characterization of chelating molecules, such as the possible testing of very numerous compositions mimicking different contamination sites or biological fluids associated therewith. In addition, the fact that this method is able to be implemented with relatively small volumes of gel or contact solution means that only small quantities of test molecules are required.

Therefore, the present invention concerns a method for identifying a molecule having chelating properties towards a radioelement, comprising steps of:

i) measuring the bioavailability of said radioelement applying a method such as previously defined wherein the solution mimicking the biological fluid comprises a molecule likely to exhibit chelating properties;

ii) measuring the bioavailability of said radioelement applying a method such as previously defined under the same conditions as at step (i), the solution mimicking the biological fluid being free of said molecule likely to exhibit chelating properties; and

iii) comparing the bioavailability obtained at step (i) with that obtained at step (ii), whereby, if the bioavailability obtained at step (i) is higher than obtained at step (ii), it is an indication that the molecule tested at step (i) has chelating properties towards said radioelement.

For said method, at steps (i) and (ii), the same conditions are used in terms of composition of the gel mimicking a contamination site, the type and amount of radioelement initially contained in this gel, composition of the solution mimicking the biological fluid, measurement technique of the radioelement contained in the solution and time t at which this measurement is performed, the only difference being the presence or absence of the molecule to be tested in the solution mimicking the biological fluid. In addition, it is possible to obtain a dose-response effect by performing step (i) in the presence of different quantities of the molecule to be tested.

Using comparable strategies, it is possible:
to compare the chelating properties of a given molecule towards different radioelements by implementing a method of the invention using gels of identical composition prepared so as each to contain an identical quantity of a given radioelement;
to compare at least two molecules having chelating properties towards one same radioelement, the comparison of the bioavailability of the radioelement obtained for each molecule allowing classification of the chelating properties thereof in relation to one another;
to compare the chelating properties of a given molecule towards a given radioelement as a function of the site contaminated by this radioelement, by comparing the bioavailability of the radioelement obtained through the use of gels of different compositions;
to compare the chelating properties of a given molecule towards a given radioelement as a function of the biological fluid through which it reaches the site contaminated by this radioelement, by comparing the bioavailability of the radioelement obtained when using solutions of different compositions in contact with a gel of same composition; and
to test different concentrations of chelating molecules.

Other characteristics and advantages of the present invention will become further apparent to skilled persons on reading the following nonlimiting examples given for illustration purposes and with reference to the appended Figures.

### BRIEF DESCRIPTION OF THE DRAWINGS

FIG. 1 illustrates the quantity of Evans Blue recovered when implementing a method of invention, as obtained after different experiments performed by two different operators.

FIG. 2 gives total α-activity of MOX, of Pu in nitrate salts and of Am in nitrate salts, recovered by implementing a method of the invention.

FIG. 3 gives total α-activity of Pu recovered from gels mimicking different contamination sites after injury, when implementing a method of the invention.

FIG. 4 gives total α-activity of Pu recovered when implementing a method of the invention on gels in contact with different media containing or not containing chelating molecules (DTPA and 3,4-LIHOPO).

### DETAILED DESCRIPTION OF PARTICULAR EMBODIMENTS

### I. Repeatability and Reproducibility of the Method of the Invention

I.1. Operating Protocol.

Two hundred and fifty mg of agarose (Type IIA; Medium EEO) are added to 10 ml of physiological saline solution (8.8 g/L NaCl, 0.37 g/L KCl) in a beaker. This mixture is weighed and heated in a microwave oven (170 W×30 s×2; 170 W×20 s×1). After cooling for 2 min, the beaker is again weighed to adjust the volume to the value of the initial volume with physiological saline.

One hundred μl of Evans Blue (0.2% in water) are added to the agarose under gentle mixing. After cooling for about 5-10 minutes, 700 μl of the agarose solution containing the Evans Blue are distributed in the wells of a culture plate (12-well plate) using a Gilson P1000 pipette, the conical tip being cut to facilitate pipetting.

Three ml of physiological saline solution are added to the gels. The plates are incubated at 37° C. in a humid atmosphere in the presence of 5% CO₂ for 2 h under slow agitation (5 rpm). Two hours after the start of incubation, the medium is taken from each well and the percentage of Evans Blue is measured by spectrophotometry at 610 nm. The gels are dissolved in formamide and optical density is measured. The concentrations of Evans Blue in the samples are calculated from a range of increasing concentrations of the colouring agent.

I.2. Results.

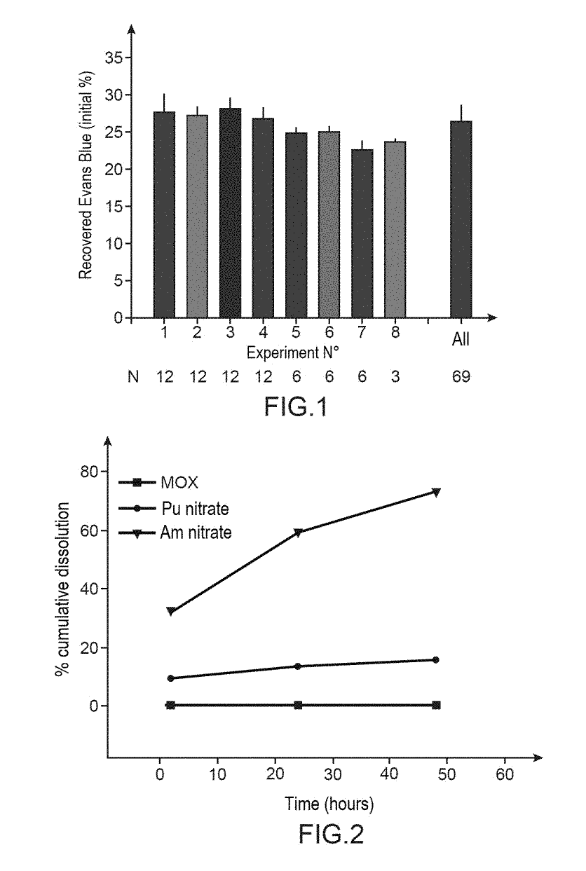

Eight experiments were performed under the same conditions over several days, and by two operators using 3 to 12 samples per experiment (FIG. 1). The data obtained show repeatability by one same operator with a coefficient of variation of about 8.6%, and reproducibility between two operators with a coefficient of variation of about 8.5%.

### II. Dissolution of Actinides

II.1. Operating Protocol.

The solutions and suspensions of actinides are prepared as follows:

MOX: 25 mg of MOX (Mixed OXide i.e. mixture of uranium and plutonium oxides) are placed in 5 ml of absolute ethanol and vigorously vortexed. Five hundred μl of this suspension are diluted 1:4 in 0.9% NaCl. An aliquot is taken to measure total α-activity via liquid scintillation.

Pu: An aliquot of plutonium solution (Pu) in 2N nitric acid (isotopic composition as % of total activity: 12.4% ²³⁹Pu, 85.6% ²³⁸Pu and 1.8% ²⁴¹Am) of about 4-5 kBq is placed in a glass liquid scintillation vial placed over a hot plate (100° C.) until complete evaporation. The Pu is then taken up in 100 μl of water or NaCl/KCl buffer. Total activity is measured by liquid scintillation.

Am: An aliquot of americium (Am) solution in 2N nitric acid of about 4-5 kBq is placed in a glass liquid scintillation vial over a hot plate (100° C.) until complete evaporation. The Am is then taken up with 100 μl of water. Total activity is measured by liquid scintillation.

A 2.5% agarose solution is prepared in physiological saline solution (8.8 g/L NaCl, 0.37 g/L KCl, pH 5.6) through the addition of 0.375 g agarose (low melting point, A11-EE0 Sigma Aldrich) to 15 ml of physiological saline solution in a beaker. The solution is heated for 30 s in a microwave oven at 250 W power, homogenized then heated 30 s, again homogenized and heated 20 s. After verifying the translucency of the solution, the volume is readjusted to 15 ml if needed.

After cooling to ambient temperature for 5 to 10 min, approximately 100 kBq of MOX, 4-5 kBq of Pu or Am in 100-150 μl are added to the 15 ml of agarose solution. The mixture is carefully homogenized. 700 μl of the mixture are placed in 3.9 cm² wells of a 12-well culture plate using an automatic P1000 pipette of which the tip of the end part has been cut away. The culture plate is placed under very slight agitation to distribute the agarose solution homogeneously over the bottom of the well. The plate is then left 10-15 min at ambient temperature, without lid to prevent condensation, to reach complete solidification of the gels.

Each condition is performed at least three times (3 identical wells).

At this step, the plate can be sealed with vinyl film and stored at +4° C. for later use (1-3 days) or used immediately.

Three ml of physiological saline solution are added to each well. The culture plate is then placed in a cell culture incubator at 37° C., in a humid atmosphere with 5% CO₂, under slow agitation (5 rpm).

Two hours after the start of incubation, the buffer of each well is taken and placed in a liquid scintillation vial. Fifteen ml of scintillator (Ultimagold) are added and total α-activity is measured by liquid scintillation (Packard counter). Three ml of buffer are added to each well and the plate is placed back in the incubator. The supernatants are collected in similar manner at 24 h and 48 h after the start of incubation.

At 48 h, the gels are very carefully recovered from the culture plates using a metallic spatula and can be placed in a Petri dish wrapped in film of Parafilm M° type to prevent drying and for direct counting by a detector. As a variant, the carefully collected gels are directly placed in liquid scintillation vials. 2 ml of 2N nitric acid are added to the vials. These are placed over a hot plate (100° C.) until complete evaporation. One ml of H₂O₂ (30%) is then added. After complete evaporation 1 ml of 2N nitric acid is added followed by 15 ml of scintillating liquid. The activity of each sample is measured i.e. the supernatants already collected and the gel.

II.2. Results.

The activities of each well, measured in the supernatants at 2 h, 24 h and 48 h are added to that of the gels. This value forms 100% of each well i.e. the initial activity deposited in the wells. The results are then expressed as activity measured in the supernatants/initial activity and are given in FIG. 2. It is to be noted that the activity at 24 h is equal to the sum of activity of the supernatant at 2 h and of the supernatant at 24 h, and that the activity at 48 h is the sum of the activity of the supernatant at 2 h, of the supernatant at 24 h and of the supernatant at 48 h.

Compared with initial activity, there is less activity derived from the MOX contained in the supernatant than from Pu or Am in nitrate salts. The latter are therefore less retained in the agarose gel than MOX: Pu and Am are therefore more bioavailable when in the form of nitrate salts than when contained in MOX.

### III. Identification of Plutonium-Retaining Compartments

III.1. Operating Protocol.

The Pu solution is prepared in the same manner as described under item II.1 above, then added to the agarose solution prepared in buffers of varying composition, as a function of the mimicked physiological compartment. A solution of agarose prepared in NaCl/KCl is used as reference.

To mimic synovial fluid (SYN): a solution of 8.8 g/L NaCl, 0.37 g/L KCl, 1.44 g/L Na₂HPO₄, 0.24 g/L KH₂PO₄, 3 g/L hyaluronic acid is used to prepare the agarose gel.

To mimic the extracellular matrix (ECM-1): a solution of 8 g/L NaCl, 0.2 g/L KCl, 1.44 g/L Na₂HPO₄, 0.24 g/L KH₂PO₄, is used to prepare the agarose gel as described under item II.1 above. After cooling at ambient temperature for 5 to 10 min, collagen (type I, from rat tail) is added to obtain a final concentration of 0.5 g/L. The pH is adjusted to 7 with acetic acid.

To mimic the extracellular matrix (ECM-2): a solution of 8 g/L NaCl, 0.2 g/L KCl, 1.44 g/L Na₂HPO₄, 0.24 g/L KH₂PO₄, 3 g/L hyaluronic acid, 1.1 g/L glucosamine is used to prepare the agarose gel. After cooling at ambient temperature for 5 to 10 min, collagen (type I, from rat tail) is added to obtain a final concentration of 0.5 g/L. The pH is adjusted to 7 with acetic acid.

To mimic cartilage (CHON): a solution of 8 g/L NaCl, 0.2 g/L KCl, 1 g/L chondroitin is used to prepare the agarose gel.

After homogenization, 4-5 kBq of Pu in 100-150 μl are added to each of the different agarose solutions. The remainder of the experiment is similar to that described under item II.1 above.

III.2. Results.

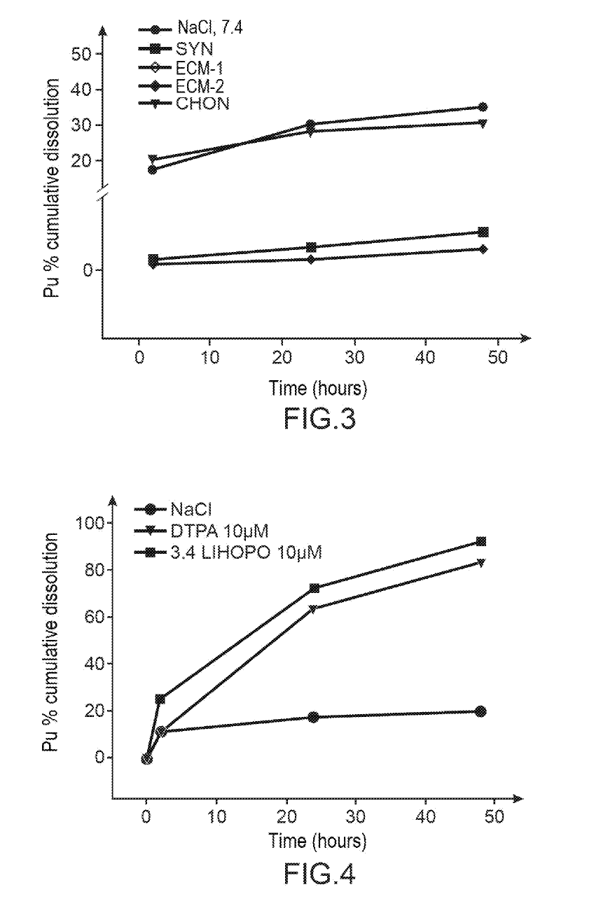

As set forth in the protocol under item II above, the activities of each well, measured in the supernatants at 2 h, 24 h and 48 h, are added to that of the gels. This value forms 100% of each well i.e. the initial activity deposited in the wells. The results are therefore expressed as activity measured in the supernatants/initial activity and are given in FIG. 3.

It follows from the mimicked compartments that there are two types of behaviour with (1) the compartments in which Pu is retained namely the synovial fluid and extracellular matrix, the two curves corresponding to ECM-1 and ECM-2 being juxtaposed, and (2) those which do not retain either Pu or the reference agarose, namely the cartilage. Therefore, Pu appears to be less bioavailable in synovial fluid and the extracellular matrix than in cartilage.

### IV. Evaluation of the Efficacy of Plutonium-Chelating Molecules

IV.1. Operating Protocol.

The Pu solution and agarose solution are prepared in the same manner as described under item II.1 above. The preparation of the Pu-containing gels is carried out as described under item II.1 above.

Incubation of the agarose gels takes place in buffers of different compositions as a function of the chelating molecule to be tested. Incubation in NaCl/KCl buffer is used as reference. A solution of diethylenetriamine pentaacetic acid (Ca-DTPA, Pharmacie centrale des armées) is diluted in NaCl/KCl to obtain a 10 μM concentration. A solution of 3,4-LIHOPO is prepared with a final concentration of 10 μM. The remainder of the experiment is similar to the description under item II.1 above.

IV.2. Results.

As set forth in the protocol under item II above, the activities of each well, measured in the supernatants at 2 h, 24 h and 48 h, are added to that of the gels. This value forms 100% of each well i.e. the initial activity deposited in the wells. The results are therefore expressed as activity measured in the supernatants/initial activity and are given in FIG. 4.

As expected, given the nature of Ca-DTPA and 3,4-LIHOPO, there is less retention of Pu in the agarose gel, Pu therefore being more bioavailable when the medium surrounding this gel comprises a chelating molecule, compared with a medium devoid thereof.

### REFERENCES

[1] Ansoborlo et al, 1999, «Review and critical analysis of available in vitro dissolution tests», Health Physics, vol. 77, pages 638-645.
[2] Davison and Zhang, 1994, «In situ speculation measurements of trace components in natural waters using thin film gels», Nature, vol. 367, pages 546-548.
[3] Klose et al, 2009, «Towards more realistic in vitro release measurement techniques for biodegradable microparticles», Pharmaceutical Research, vol. 26, pages 691-699.
[4] Hoang Thi et al, 2010, «Bone implants modified with cyclodextrin: Study of drug release in bulk», International Journal of Pharmaceutics, vol. 400, pages 74-85.
[5] Chaibva and Walker, 2007, «The comparison of in vitro release methods for the evaluation of oxytocin release from Pluronic® F127 parenteral formulations», Dissolution Technologies, vol. 14, pages 15-25.
[6] Marques et al, 2011, «Simulated biological fluids with possible application in dissolution testing», Dissolution Technologies, vol. 18, pages 15-28.
[7] Zammit et al, 1994, «Effects on fluid and Na+ flux of varying luminal hydraulic resistance in rat colon in vivo», Journal of Physiology, vol. 477, pages 539-548.

What is claimed is:

1) Method for predicting the bioavailability of a radioelement in a living animal body further to contamination of this body by said radioelement, comprising the steps of:
a) preparing a gel in which said radioelement is uniformly distributed, said gel mimicking the contamination site in said living animal body;
b) placing the gel prepared at said step (a) in contact with a solution mimicking a biological fluid associated with the contamination site in said living animal body, then placing the whole under agitation; and
c) measuring, at a time t, the amount of said radioelement in said solution, said measurement allowing prediction of the bioavailability of said radioelement in said living animal body.

2) The method according to claim 1, wherein said radioelement is an actinide.

3) The method according to claim 1, wherein the gel is prepared at step said (a) from at least one compound having colloidal properties, a solution called gel solution and at least one radioelement.

4) The method according to claim 3, wherein said compound having colloidal properties is a polysaccharide selected in particular from the group consisting of agarose, sucrose, sepharose, chitosan, xanthan, carrageenan, dextran, agar, alginate or mixtures thereof.

5) The method according to claim 3, wherein the ratio between compound(s) having colloidal properties (weight expressed in g)/gel solution (volume expressed in ml) is between 1 and 5%, typically between 2 and 4%.

6) The method according to claim 3, wherein said gel solution is an aqueous solution e.g. physiological saline solution optionally completed with one or more organic or inorganic salts selected from the group consisting of sodium chloride (NaCl), potassium chloride (KCl), sodium bicarbonate (NaHCO₃), sodium phosphate (Na₃PO₄), sodium hydrogen phosphate (Na₂HPO₄), sodium dihydrogen phosphate (NaH₂PO₄), potassium hydrogen phosphate (K₂HPO₄), potassium dihydrogen phosphate (KH₂PO₄), hydrochloric acid (HCl), magnesium chloride (MgCl₂), calcium chloride (CaCl₂), sodium sulfate (Na₂SO₄), sodium hydroxide (NaOH), potassium hydroxide (KOH), sodium acetate (CH₃COONa), sodium tartrate (Na₂C₄H₄O₆), sodium lactate (C(OH)(CH₃)COONa), sodium pyruvate (CH₃C(O)COONa), sodium citrate (Na₃C₆H₅O₇) and citric acid (C₆H₈O₇).

7) The method according to claim 3, wherein the gel comprises one or more compounds selected from the group consisting of pepsin, lecithin, glycine, glucose, lysozyme, albumin, sodium taurocholate, maleic acid, transferrin, ferritin, fibrin, hyaluronic acid, glucosamine, fibronectin, laminin, mucin, keratins, collagens, chondroitin, osteopontin, hydroxyapatite and glutamic acid.

8) The method according to claim 3 wherein, at step (a), the radioelement is added in the form of a suspension or of a solution to the gel solution containing at least one compound having colloidal properties.

9) The method according to claim 3, characterized in that the solution of said step (b) is an aqueous solution comprising one or more organic or inorganic salts selected from the group consisting of sodium chloride (NaCl), potassium chloride (KCl), sodium bicarbonate (NaHCO₃), sodium phosphate (Na₃PO₄), sodium hydrogen phosphate (Na₂HPO₄), sodium dihydrogen phosphate (NaH₂PO₄), potassium hydrogen phosphate (K₂HPO₄), potassium dihydrogen phosphate (KH₂PO₄), hydrochloric acid (HCl), magnesium chloride (MgCl₂), calcium chloride (CaCl₂), sodium sulfate (Na₂SO₄), sodium hydroxide (NaOH), potassium hydroxide (KOH), sodium acetate (CH₃COONa), sodium tartrate (Na₂C₄H₄O₆), sodium lactate (C(OH)(CH₃)COONa), sodium pyruvate (CH₃C(O)COONa), sodium citrate (Na₃C₆H₅O₇) and citric acid (C₆H₈O₇).

10) The method according to claim 3, characterized in that said gel solution and said solution used at step (b) comprise one or more salts, the same or different.

11) The method according to claim 3, characterized in that the solution of said step (b) further comprises one or more compounds selected from the group consisting of pepsin, lecithin, glycine, glucose, lysozyme, albumin, sodium taurocholate, maleic acid, transferrin, ferritin and fibrin.

12) The method according to claim 3, characterized in that the solution of said step (b) is prepared in an incubator with agitation having a temperature of approximately 37° C., the controlled composition of the atmosphere being humid air comprising 5% CO₂.

13) Use of a method according to claim 1, to identify a molecule having chelating properties towards a given radioelement and/or to characterize the chelating properties of a molecule.

14) Method for identifying a molecule having chelating properties towards a radioelement, comprising the steps of:
i) measuring the bioavailability of said radioelement with the method according to claim 1, wherein the solution mimicking the biological fluid comprises a molecule likely to exhibit chelating properties;
ii) measuring the bioavailability of said radioelement with the method according to claim 1 under the same conditions as at step (i), the solution mimicking the biological fluid being free of said molecule likely to exhibit chelating properties; and
iii) comparing the bioavailability obtained at step (i) with that obtained at step (ii) whereby, if the bioavailability obtained at step (i) is higher than obtained at step (ii), it is an indication that the molecule tested at step (i) has chelating properties towards said radioelement.

15) The method according to claim 14, characterized in that at said steps (i) and (ii), the same conditions are used in terms of composition of the gel mimicking a contamination site, type and amount of radioelement initially contained in this gel, composition of the solution mimicking the biological fluid, technique for measuring the radioelement contained in the solution, and time t at which this measurement is performed, the only difference being the presence or absence of the molecule to be tested in the solution mimicking the biological fluid.
